(19) United States
(12) Patent Application Publication
(10) Pub. No.: US 2025/0041119 A1
(43) Pub. Date: Feb. 6, 2025

(54) METHOD FOR PROVIDING CONTROL DATA FOR AN OPHTHALMOLOGICAL LASER OF A TREATMENT APPARATUS

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/790,429

(22) Filed: Jul. 31, 2024

(30) Foreign Application Priority Data
Aug. 3, 2023 (DE) 10 2023 120 647.8

(51) Int. Cl. A61F 9/008

(52) U.S. Cl. CPC A61F 9/008; A61F 2009/00859; A61F 2009/00872

(57) ABSTRACT

The invention relates to apparatuses and methods for providing control data for an ophthalmological laser (12) of a treatment apparatus (10). The method includes ascertaining (S10) correction data for a planned refraction correction of a visual disorder of a cornea (16) from predetermined examination data, wherein the correction data includes a correction for a sphere value, a cylinder value and an axis value; determining (S12) adapted correction data based on a nomogram and the ascertained correction data, wherein the correction of the sphere value and/or the cylinder value and/or the axis value is adapted to the used treatment apparatus (10) by the nomogram, such that a refraction correction with the adapted correction data corresponds to the planned refraction correction, wherein the nomogram is ascertained by preceding treatment results with identical treatment apparatuses; and providing (S14) the control data, which is based on the adapted correction data.

### FIELD

The invention relates to a method for providing control data for an ophthalmological laser of a treatment apparatus. Furthermore, the invention relates to a control device, which is configured to perform the method, to a treatment apparatus with such a control device, as well as to a computer program comprising commands, which cause the treatment apparatus to execute the method. In addition, a computer-readable medium is provided, on which the computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device can for example be formed such that laser pulses effect a photodisruption and/or ablation in a focus area situated within the organic tissue to remove a tissue, in particular a tissue lenticule, from the cornea.

In treating a cornea, it can occur that an achieved correction deviates from a planned correction. In particular, this can be based on properties of the environment, in which the treatment apparatus is located, on a demography, on treatment or diagnostic differences by the physician and/or on a systematic distortion of used appliances.

### SUMMARY

Therefore, it is the object of the invention to provide control data for controlling the ophthalmological laser and/or the treatment apparatus, which generates an improved correction.

This object is solved by the independent claims. Advantageous embodiments of the invention are disclosed in the dependent claims, the following description as well as the Figures.

The invention is based on the idea that a nomogram is created, which includes planned and achieved corrections, in particular of sphere, cylinder and axis values. In the treatment planning, the planned correction data required for the treatment can then be inferred from the nomogram based on the achieved correction data.

An aspect of the invention relates to a method for providing control data for an ophthalmological laser of a treatment apparatus, wherein the method comprises the following steps performed by a control device. An appliance or an appliance component, in particular a computer or processor, which can automatically or semi-automatically perform the following steps, is to be understood by a control device.

In the method, ascertaining correction data for a planned refraction correction of a visual disorder of a cornea from predetermined examination data is effected, wherein the correction data includes a correction for a sphere value, a cylinder value and an axis value. Furthermore, determining adapted correction data based on a nomogram and the ascertained correction data is effected, wherein the correction of the sphere value and/or of the cylinder value and/or of the axis value is adapted to the used treatment apparatus by the nomogram, such that a refraction correction with the adapted correction data corresponds to the planned refraction correction, wherein the nomogram is ascertained by preceding treatment results with identical treatment apparatuses. Finally, providing the control data, which is based on the adapted correction data, is effected.

In other words, an achieved refraction correction can deviate from a planned refraction correction due to characteristics of the treatment apparatus. This deviation can be compensated for by the nomogram in that the initially ascertained correction data is adapted such that the planned refraction correction is obtained as the correction.

Hereto, the initial correction data for correcting a refraction of the cornea can first be ascertained from predetermined examination data. The predetermined examination data can for example originate from a manifest or subjective refraction measurement, in particular by a phoropter. Alternatively or additionally, the examination data can include wavefront measurements and/or topography measurements of the cornea. The refraction correction for compensating for a visual disorder can include at least a sphere value, a cylinder value and an axis value.

From the nomogram, which may rely on the basis of preceding treatments with similar treatment apparatuses, for example the same treatment apparatus, treatment results of these treatment apparatuses can be present together with an initially planned correction. By the control device, the initially planned correction data can then be compared to achieved treatment results to determine via the nomogram, which initial correction data can be used for achieving these treatment results. This means that an initially planned sphere value may be compared to an achieved sphere value and an adapted sphere value is determined via the nomogram, furthermore an achieved cylinder value is determined to an adapted cylinder value via the nomogram, and an axis value is determined to an adapted axis value via the nomogram.

Finally, the adapted correction data, which in particular includes the adapted sphere, cylinder and axis values, can be provided in the form of control data, wherein the treatment apparatus and/or the ophthalmological laser can be controlled by the control data.

The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

In particular, the nomogram can include changes in the manifest refraction, changes in the cycloplegic refraction, changes in the autorefraction, changes in the aberrometric refraction at a fixed analysis diameter (and reference center), changes in the aberrometric refraction for a maximum pupil size, changes in the keratometric refractive power or similar topographic metrics, with the aim to obtain an optimized nomogram, from which the adapted values can be ascertained for achieving the refraction correction.

By the invention, the advantage arises that an improved refraction correction can be performed, since properties of the similar or identical treatment apparatus can be compensated for.

The invention also includes embodiments, by which additional advantages arise.

In an embodiment the nomogram is ascertained by preceding treatment results with the same treatment apparatus. This means that the nomogram is specifically tailored to the used treatment apparatus. In particular, a nomogram can be created for each laser of the treatment apparatus. Hereto, after a preset number of treatments, for example after ten treatments, the nomogram can be approved for the use, whereby the initially planned correction data can be adapted. Hereby, the advantage arises that the nomogram considers characteristic properties of the used treatment apparatus.

In another embodiment the nomogram is automatically updated. In other words, treatment results or postoperative data can be restored to the treatment apparatus and/or the control device, in particular using artificial intelligence, to complete the nomogram and/or to improve an accuracy.

For example, it is provided that the preceding treatment results are temporally weighted in the nomogram. In particular, it can be provided that more recent treatment results are higher weighted than older treatment results. Thus, it can be ensured among other things that a temporal change of the laser and/or of the treatment apparatus is detected, whereby improved adapted correction data can be created.

In another embodiment the nomogram is newly generated after preset periods of time and/or after a preset number of new treatment results. Thereby, it can in particular be avoided that outliers have a too strong weighting in the nomogram. In addition, it can be reacted to changes of the laser and/or the treatment apparatus in an improved manner. The preset periods of time, after which the nomogram is newly generated, can for example be dependent on seasons such that environmental conditions can be taken into account in an improved manner. The preset number, after which the nomogram can be newly generated, can for example occur after 100 or 200 treatments with the treatment apparatus.

In another embodiment the nomogram is based on a linear correlation between a planned refraction correction and an achieved refraction correction. This means that a regression between the planned and the achieved refraction correction can be performed, wherein the value for the adapted correction parameters can be determined by this linear balancing function. In particular, this can respectively be performed for the sphere value, cylinder value and axis value. In particular, the nomogram can be present in different coordinate systems.

For example, the nomogram can be provided as a sphere and negative cylinder, wherein the adapted correction values thus arise to:

$${S}_{conv} = {S}_{orig} + {C}_{orig}$$

$${C}_{conv} = - {C}_{orig}$$

$${A}_{conv} = mod ( {A}_{orig} + 90 , 180 )$$

wherein S_{conv} is the adapted sphere value, S_{orig} is the initially planned sphere value, C_{orig} is the initially planned cylinder value, C_{conv} is the adapted cylinder value, A_{conv} is the adapted axis value and A_{orig} is the initially planned axis value, and mod (x, y) is a modulo function with inputs x, y where mod (x, y) returns the remainder of the division of x/y. Alternatively or additionally, the nomogram can also be represented as a sphere and positive cylinder.

In an alternative configuration, the nomogram can be provided with the aid of the spherical equivalent and the astigmatism (axis value). Herein, the achieved change of the spherical equivalent can be correlated with the planned change, and the absolute magnitude of the achieved cylindrical change (vectorial analysis) with the planned change. Furthermore, effects of a cyclotorsion of a residual astigmatism can for example also be taken into account.

In a further alternative configuration, the nomogram can be provided based on the principal meridians. Herein, the sphere, cylinder and axis value (spherocylindrical values; SCA) can be converted to the principal meridians, in particular by:

$${Cyl}_{1} = {S}_{orig}$$

$${Cyl}_{2} = {S}_{orig} + {C}_{orig} .$$

In a further alternative configuration, the nomogram can be provided based on a cardinal and oblique astigmatism according to the Jackson cross cylinder, in which the sphere, cylinder and axis values are represented in [M, C+, Cx]. In particular, further power vector notations can also be used.

For some strategies, the sphere, cylinder and axis values can be represented in [M, C+, Cx], wherein this notation is based on mathematically independent components (vector character). In particular, it is also possible that the nomogram is constructed of multiple variants and the statistically most probable variant is selected. Other types of nomograms can be calculated, if the availability of data is sufficiently great, via a distance metric, for example:

$$Distance = \sqrt{{( {( {M}_{1} - {M}_{2} )}^{2} + {( C {+}_{1} - C {+}_{2} )}^{2} + {Cx}_{1} - {Cx}_{2} )}^{2} )}$$

between the planned correction and the correction in the database. Herein, one can use the most similar with the lowest distance or all with a distance below a tolerance, to create the nomogram only from this subset.

In another embodiment the nomogram is additionally provided depending on patient parameters and/or environmental parameters and/or laser parameters. This means that these parameters can be ascertained in the present treatment and a nomogram is retrieved with treatment results of treatments, in which these parameters were also present. For example, the parameters can include: a laterality, that is information about a right or left eye, and/or an achieved treatment result of the other eye and/or a target refraction and/or a preoperative CDVA (best corrected vision, for example like with glasses) and/or an amblyopia (value for a “bad” eye in case of a severe difference between the eyes) and/or a time after the treatment results have been ascertained, and/or a wavefront aberration and/or a UDVA (uncorrected vision) and/or an asphericity and/or a planned transition zone and/or a planned optical zone and/or a repetition rate of the laser and/or a treatment duration and/or a water content of the cornea and/or an adaptation of the lens to sharpen an image and/or a patient age and/or a corneal curvature and/or cross-effects, for example if a sphere is treated and a cylindrical component additionally arises, and/or a neuronal adaptation of the patient and/or an air humidity and/or an air pressure.

In another embodiment the nomogram is determined based on achieved refraction values in preceding treatments and/or based on achieved topographic changes of the cornea in preceding treatments. In other words, the sphere, cylinder and axis value can be determined either by measurements of the refraction, for example of a subjective manifest refraction by a phoropter, or from measurements of the morphology, for example topography measurements.

For example, it is provided that at least two nomograms are provided, one based on the refraction values and the other based on the topographic change, wherein an adapted correction data range is determined, with limit values, which are provided by the respective nomogram. This means that at least two nomograms are present, and values, which can be provided from these nomograms, can provide limit values for a range, in which the adaptation can occur. Herein, a nomogram can be provided based on refraction values, which are subjectively present for a patient, and the other one by objective measurements, in particular topography measurements, which indicate a curvature of the cornea. Thus, it can for example be weighted if a treatment result is to provide an improved subjective refraction or an improved objective refraction. In particular, it can be provided that if the initially planned correction data is in the provided range, an adaptation cannot occur.

In another embodiment a warning message is generated if the adapted correction data deviates from the initial correction data by a preset limit value and/or if a statistic of the nomogram for the correction data is below a threshold value. The warning message may be, in particular optically and/or acoustically, output to a user via a user interface, if the adapted correction data deviates from the initial correction data by a preset limit value. For example, a relative limit value can be preset, in particular at 25 percent. Alternatively or additionally, a warning message can be generated and output if a statistic of the nomogram is below a threshold value. This means that either the entire nomogram has too little data to derive statistically significant values therefrom, and/or a range of the nomogram, in which the refraction correction occurs, can have no or too few values, whereby a warning message is output. Thus, at least ten treatments, for example 20, can be provided before the nomogram can be used without warning message.

A further aspect relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially separate a predefined corneal volume with predefined interfaces of a human or animal eye by optical breakdown, in particular at least partially separate it by photodisruption and/or to ablate corneal layers by (photo) ablation and/or to effect a laser-induced refractive index change in the cornea and/or the eye lens and/or to increase a crosslinking of the cornea.

In another embodiment of the treatment apparatus according to the invention, the laser can be suitable to emit laser pulses in a wavelength range between 300 nm and 1400 nm, for example between 900 nm and 1200 nm, at a respective pulse duration between 1 fs and 1 ns, for example between 10 fs and 10 ps, and a repetition frequency of greater than 10 kilohertz (kHz), for example between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakdown can be spatially narrowly limited such that a high incision accuracy is allowed in the generation of the interfaces. In particular, the range between 700 nm and 780 nm can also be selected as the wavelength range.

In another embodiment of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above-mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment.

FIG. 2 depicts a schematic method diagram for providing control data according to an exemplary embodiment.

FIG. 3 depicts an exemplary diagram with an achieved refraction correction and a planned refraction correction for providing a nomogram.

In the figures, identical or functionally identical elements are provided with the same reference characters.

### DETAILED DESCRIPTION

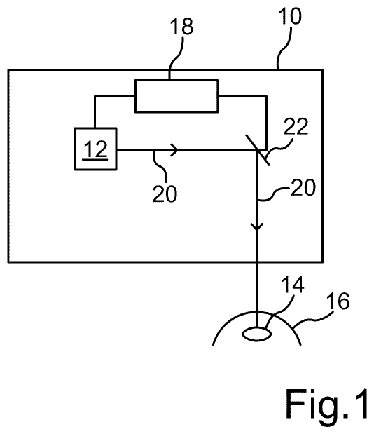

FIG. 1 shows a schematic representation of a treatment apparatus 10 with an ophthalmological laser 12 for removing a tissue 14 from a human or animal cornea 16 by photodisruption and/or ablation. For example, the tissue 14 can represent a lenticule or also volume body, which can be separated from the cornea 16 with the eye surgical laser 12 for correcting a visual disorder. A geometry of the tissue 14 to be removed can be preset by correction data, which is provided to a control device 18, in particular in the form of control data, such that the laser 12 emits pulsed laser pulses in a pattern predefined by the control data into the cornea 16 of the eye, to remove the tissue 14. Alternatively, the control device 18 can be a control device 18 external with respect to the treatment apparatus 10.

Furthermore, FIG. 1 shows that the laser beam 20 generated by the laser 12 can be deflected towards the cornea 16 by a beam deflection device 22 such as for example a rotation scanner, to remove the tissue 14. The beam deflection device 22 can also be controlled by the control device 18 to remove the tissue 14.

In particular, the illustrated laser 12 can be a photodisruptive and/or photoablative laser, which is formed to emit laser pulses in a wavelength range between 300 nanometers and 1400 nanometers, for example between 700 nanometers and 1200 nanometers, at a respective pulse duration between 1 femtosecond and 1 nanosecond, for example between 10 femtoseconds and 10 picoseconds, and a repetition frequency of greater than 10 kilohertz, for example between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

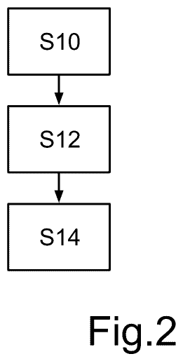

In the refraction correction by removing the tissue 14 from the cornea 16, it can occur that a planned refraction correction deviates from an achieved refraction correction. For example, this can be dependent on characteristics of the treatment apparatus, in particular a location and/or environmental conditions of the treatment apparatus 10. In order to consider these effects and to provide improved control data for refraction correction, the method shown in FIG. 2 can be performed by the control device 18.

In a step S10, initial correction data for a planned refraction correction can be determined from predetermined examination data, wherein the correction data comprises a correction for at least a sphere value, a cylinder value and an axis value. The predetermined examination data may be ascertained from a subjective measurement, for example by a phoropter, by which the corresponding sphere, cylinder and axis values for the treatment can be determined.

In a step S12, it can then be ascertained based on a predetermined nomogram how the initially determined sphere, cylinder and axis values are to be adapted to achieve the originally planned refraction correction. Herein, the nomogram may be adapted to the used treatment apparatus, wherein preceding treatment results, which have an achieved refraction correction with the treatment apparatus 10, with the originally planned refraction correction can be provided in the nomogram.

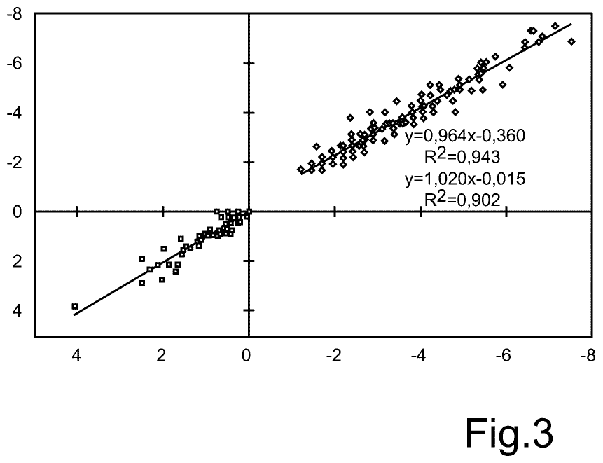

For example, an exemplary representation of achieved refraction corrections (ordinate) with the corresponding planned refraction corrections (abscissa) is represented in FIG. 3, wherein the values top right in the negative range can represent the sphere values, and the values bottom left in the positive range can represent the cylinder values. A corresponding diagram may also be present for the axis values. In particular, other notations of the sphere, cylinder and axis values can also be provided.

The provided nomogram may be automatically updated in that for example artificial intelligence restores the treatment results or postoperative data to the treatment apparatus 10 and/or the control device 18 and complements or adapts the nomogram. Herein, it can for example be provided that the newly recorded treatment results are temporally higher weighted than old treatment results due to the timeliness. In particular, it can be provided that the nomogram is newly created after preset periods of time and/or after a preset number of new treatment results. Alternatively, treatment results, which have exceeded a preset period of time, can also be removed from the previous nomogram such that the nomogram can always be kept up to date.

In particular, it can also be provided that the nomogram is provided based on achieved refraction values, which are determined by subjective measurement methods, and a second nomogram is additionally provided, which is determined with objective measurement methods, in particular morphological measurements. Then, correction ranges can be determined therefrom, which have limit values, which are provided by the respective nomograms.

The initially determined correction data can then be adapted depending on the nomogram, wherein the adapted correction data can be provided to the control device 18 in the form of control data for controlling the laser 12 and/or the beam deflection device 22 in a step S14. This means that the treatment apparatus 10 can be controlled with the control data for refraction correction of the visual disorder.

For example, it can be provided that a warning message is generated if the adapted correction data deviates from the initial correction data by a preset value and/or if statistics of the nomogram, that is captured treatment values, is below a threshold value.

Overall, the examples show how an automatic provision of nomograms can be achieved.

1. A method for providing control data for an ophthalmological laser of a particular treatment apparatus, wherein the method comprises the following steps performed by a control device:
ascertaining initial correction data for a planned refraction correction of a visual disorder of a cornea from predetermined examination data, wherein the initial correction data includes a correction for a sphere value, a cylinder value and an axis value;
determining adapted correction data based on a nomogram and the ascertained initial correction data, wherein the correction of the sphere value and/or the cylinder value and/or the axis value is adapted based on the nomogram for the particular treatment apparatus, such that a refraction correction with the adapted correction data corresponds to the planned refraction correction, wherein the nomogram is ascertained by preceding treatment results performed by a treatment apparatus identical to the particular treatment apparatus; and
providing the control data, which is based on the adapted correction data.

2. The method according to claim 1, wherein the treatment apparatus identical to the particular treatment apparatus is the particular treatment apparatus.

3. The method according to claim 1, wherein the nomogram is automatically updated.

4. The method according to claim 3, wherein the preceding treatment results are temporally weighted in the nomogram.

5. The method according to claim 3, wherein the nomogram is newly generated after preset periods of time and/or after a preset number of new treatment results.

6. The method according to claim 1, wherein the nomogram is based on a linear correlation between planned refraction corrections and achieved refraction corrections of the preceding treatment results.

7. The method according to claim 1, wherein the nomogram is additionally provided depending on patient parameters and/or environmental parameters and/or laser parameters.

8. The method according to claim 1, wherein the nomogram is determined based on achieved refraction values in preceding treatments and/or based on achieved topographic changes of the cornea in preceding treatments.

9. The method according to claim 8, wherein at least two nomograms are provided, one based on the achieved refraction values and another one based on the achieved topographic changes, wherein an adapted correction data range is determined with limit values, which are provided by the respective nomogram.

10. The method according to claim 1, wherein a warning message is generated if the adapted correction data deviates from the initial correction data by a preset limit value and/or if statistics of the nomogram for the initial correction data is below a threshold value.

11. A control device configured to perform the method according to claim 1.

12. A treatment apparatus with at least one eye surgical laser for separation of a corneal volume with predefined interfaces of a human or animal eye by optical breakdown and at least one control device according to claim 11.

13. (canceled)

14. A computer-readable medium for storing a computer program, the computer program comprising commands which, when executed, cause a treatment apparatus to execute the method according to claim 1.
